(19) United States
(12) Patent Application Publication
(10) Pub. No.: US 2020/0018773 A1
(43) Pub. Date: Jan. 16, 2020

(54) FLUID OPERATION CELL WITH ON-CHIP ELECTRICAL FLUID OPERATION COMPONENTS

(71) Applicant: HEWLETT-PACKARD DEVELOPMENT COMPANY, L.P., Spring, TX (US)

(72) Inventors: Ning GE, Palo Alto, CA (US); David GEORGE, Palo Alto, CA (US); Helen A. HOLDER, Palo Alto, CA (US)

(73) Assignee: Hewlett-Packard Development Company, L.P., Spring, TX (US)

(21) Appl. No.: 16/335,738

(22) PCT Filed: Oct. 10, 2016
(86) PCT No.: PCT/US2016/056274

§ 371 Date: Mar. 22, 2019

(51) Int. Cl. G01N 35/00; G16H 40/63

(52) U.S. Cl. CPC G01N 35/00069; G16H 40/63; G01N 2035/1034; G01N 2035/00237; G01N 2035/00346; G01N 2035/00158

(57) ABSTRACT

In one example in accordance with the present disclosure, a fluid operation cell is described. The fluid operation cell includes a micro-electro-mechanical housing. The housing includes an inlet and an outlet through which fluid flows and electrodes disposed on an interior of the housing to couple to a controller. The fluid operation cell also includes a silicon-based substrate disposed inside, and electrically coupled to, the housing. The substrate includes an on-chip electrical fluid operation component formed thereon. The electrical fluid operation component uses an electrical signal to operate on bypassing fluid. The fluid operation cell includes a dedicated address to be individually activated by the controller.

### BACKGROUND

Microfluidic systems are used to perform different operations on small volumes of fluid. For example, microfluidic systems can move, mix, separate, and perform fluid analysis of different types of fluids. Such systems can be used in the medical industry, for example to analyze DNA, detect pathogens, perform clinical diagnostic testing, and aiding in synthetic chemistry. The systems can also be used in other industries.

### BRIEF DESCRIPTION OF THE DRAWINGS

The accompanying drawings illustrate various examples of the principles described herein and are part of the specification. The illustrated examples are given merely for illustration, and do not limit the scope of the claims.

FIG. 1 is an isometric view of an enclosed fluid operation cell with on-chip electrical fluid operation components, according to an example of the principles described herein.

FIG. 2 is a segmented isometric view of an interior of a fluid operation cell with on-chip electrical fluid operation components, according to an example of the principles described herein.

FIG. 3 is a diagram of a fluid operation system with a fluid operation cell with on-chip electrical fluid operation components, according to an example of the principles described herein.

FIG. 4 is a schematic of the fluid operation system with a fluid operation cell with on-chip electrical fluid operation components, according to an example of the principles described herein.

FIG. 5 is a block diagram of a computing system for controlling a fluid operation cell with on-chip electrical fluid operation components, according to an example of the principles described herein.

Throughout the drawings, identical reference numbers designate similar, but not necessarily identical, elements.

### DETAILED DESCRIPTION

Microfluidic microelectromechanical Systems (MEMS) systems are used to perform different operations on small volumes of fluid. For example, microfluidic systems can move, mix, separate, and perform fluid analysis of different types of fluids. Such systems can be used in the medical industry, for example to analyze DNA, detect pathogens, perform clinical diagnostic testing, and aiding in synthetic chemistry. Other industries as well rely on microfluidic structures. Such microfluidic structures can be used in engineering, physics, chemistry, biochemistry, nanotechnology, and biotechnology fields. While such microfluidic devices have allowed for expansive studies of fluids at a microscopic level, certain characteristics have limited their more complete integration. MEMS microfluidic systems offer increased functionality at a reduced cost. For example, MEMS-based microfluidics can operate at smaller volumes (i.e. microliters, nanoliters, picoliters, and femtoliters). MEMS-based systems also can be manufactured to smaller sizes, consume less energy and provide manufacturing operations such as film forming, doping, lithography, and etching that improve the manufacturing and operating costs.

For example, many microfluidic structures are formed in two-dimension planer substrates. That is, the fluid flows in either an x-direction or a y-direction. Moreover, the fabrication of these two-dimensional structures is regularly performed in a single operation. Accordingly, a two-dimensional surface and the fabrication process thereof prevents a more full implementation of the microfluidic structure as the two-dimensional aspect and fabrication process limit the options regarding fluid operational structures.

Accordingly, the present specification describes microfluidic cells that are modular in that they can be combined with other microfluidic cells to form a complex microfluidic system. As the cells are modular, cells with different functionality can be combined in different configurations to carry out specific microfluidic operations or to perform multiple microfluidic operations. Moreover, the microfluidic cells as described herein incorporate on-chip electrical control circuitry. The on-chip electrical control circuitry facilitates addressing, signal encoding and decoding, analog-to-digital conversion, digital-to-analog conversion, and transimpedance amplification. These on-chip electrical control circuits interact with a central controller to perform various control functions. The on-chip electrical control circuitry also performs fluidic operations such as sensing, actuation, etc. Some microfluidic cells have not incorporated control circuits into the microfluidic structures and relied entirely on fluid mechanic properties to operate on a fluid. However, the microfluidic structures of the present specification include electrical fluid operation components such that a greater variety of functions such as sensing, heating, mixing, pumping, and performing spectroscopic analysis can be performed on the fluid.

Specifically, the present specification describes a fluid operation cell. The cell includes a microelectromechanical housing. The housing includes passages through which fluid flows and electric traces disposed on an interior of the housing to couple to a controller. The cell also includes a silicon-based substrate disposed inside, and electrically coupled to, the housing. The substrate includes an on-chip electrical fluid operation component formed thereon. The electrical fluid operation component uses an electrical signal to operate on bypassing fluid. The fluid operation cell includes a dedicated address to be individually activated by the controller.

The present specification also describes a fluid operational system. The system includes a number of fluid operation cells. A fluid operation cell includes 1) a housing having passages through which fluid flows, 2) an electrical fluid operation component disposed within the housing, and 3) a connection system to interconnect with other of the number of fluid operation cells. Each fluid operation cell is also uniquely addressable. The system also includes a controller to: 1) select at least one fluid operation cell, 2) activate the selected at least one fluid operation cell, and 3) receive data from the selected at least one fluid operation cell.

The present specification also describes a computing system. The computing system includes a processor and a machine-readable storage medium communicatively coupled to the processor. The machine-readable storage medium includes an instruction set stored on the machine-readable storage medium to cooperate with the processor to: 1) select at least one of a number of modular fluid operation cells, 2) activate the selected at least one modular fluid operation cell, and 3) receive data from the selected at least one modular fluid operation cell.

In one example, using such a fluid operation cell 1) can be used across a wide variety of industries; 2) provides a robust microfluidic system due to the modular nature of the devices; 3) provides additional functionality through the inclusion of electrical fluid operation components; and 4) can be tailored to perform any, and any number, of microfluidic operations. However, it is contemplated that the devices disclosed herein may address other matters and deficiencies in a number of technical areas.

As used in the present specification and in the appended claims, the term “electrical fluidic operation component” refers to a component of the fluid operation cell that operates on the fluid. Specifically, an electrical signal is used to perform an operation on the bypassing fluid, Examples, of such electronic fluidic operation components include a fluid movement component, a fluid heating component, a fluid sensing component, a fluid level sensing component, a fluid separation component, a fluid mixing component, a fluid filtering component and a fluid agitation component. However, other examples of such fluidic operation components may be implemented in accordance with the principles described herein.

As used in the present specification and in the appended claims, the term “a number of” or similar language is meant to be understood broadly as any positive number including 1 to infinity.

FIG. 1 is an isometric view of an enclosed fluid operation cell (100) with on-chip electrical fluid operation components disposed therein, according to an example of the principles described herein. In general, the fluid operation cell (100) is used to perform any number of fluid operations on bypassing fluid. For example, the fluid operation cell (100) could determine a type of fluid or compounds intermixed with the fluid. The fluid operation cell (100) could also perform fluid mixing, fluid separation, fluid sensing, fluid level sensing, and fluid heating. The number of operations that could be performed are various and different industries may have different operations that can be performed by the fluid operation cell (100). For example, within the medical industry operations include DNA analysis, pathogen detection, clinical diagnostic testing and synthetic chemistry,

Accordingly, the fluid operation cell (100) includes a microelectromechanical housing (102). That is, the housing may have mechanical and electrical functionality built in as is described below. The housing retains the various components of the fluid operation cell (100) and receives the fluid to be operated on. Accordingly, the fluid operation cell (100) includes any number of passages (104) to allow the ingress and egress of fluid from the housing (102). The passages (104) may be used as inlet or outlets of fluid. For example, a first passage (104-1) may be an inlet and another passage (104-2) may be an outlet. At different times, the different passages (104) may switch between operating as an inlet and operating as an outlet. In some examples, certain passages (104) on the housing (102) may be specifically designated as inlets and others may be designated as outlets. For example, inlets may be one size and outlets another. However, the mechanical connection system described below may allow for such size difference between a joined inlet and outlet.

While FIG. 1 depicts two passages (104), one as an inlet and one as an outlet, that are disposed on a front and side surface, the housing (102) may include any number of passages (104) on any number of surfaces. These passages (104) interface with passages (104) on other fluid operation cells (100) to allow passage of a fluid through the array. The passages (104) can be disposed on any surface, fluid operation cells (100) within an array can be arranged and aligned along any surface relative to other fluid operation cells (100). For example, the fluid operation cells (100) could be arranged in three-dimensional structure. An array of fluid operation cells (100) form a microfluidic operational system that can perform various types, and any number of, microfluidic operations on a subject fluid.

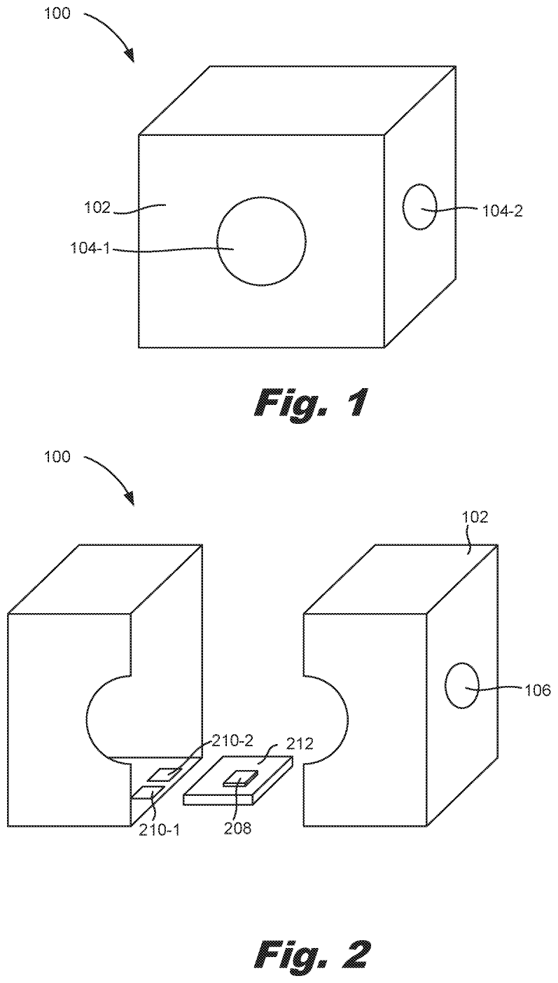

In some examples, as depicted in FIG. 2, the housing (102) may be formed of two halves that are joined together. These halves may be formed using any number of methods including injection molding. In other examples, the housing (102) is a printed housing that is formed during an additive manufacturing process. To carry out such fluidic operations, a number of components are disposed within the housing (102). In yet another example, the housing (102) may be formed by laser direct structuring (LDS) where a thermoplastic material that is doped with a metal-plastic additive activated by a laser. In LDS, a single-component is injection molded. A laser then writes the course of a circuit trace on the plastic. That is, where the laser beam hits the plastic, the metal additive forms a micro-rough track.

The substrate (212) disposed on the inside may be formed by a CMOS or semiconductor process. The substrate (212) then goes through a MEMS and packaging process and assembled with the housing (102).

Accordingly, FIG. 2 is a segmented isometric view of an interior of a fluid operation cell (100) with on-chip electrical fluid operation components (208), according to an example of the principles described herein. As will be discussed in detail below, the on-chip electrical fluid operation components (208) perform fluidic operations on the fluid using electrical signals. Accordingly, the fluid operation cell (100) includes electric traces (210) to route a control signal from a controller to the electrical fluid operation components (208). Specifically, the electric traces (210) are disposed on an interior of the housing (102) and interface with electrical contacts on a substrate (212) on which the electrical fluid operation components (208) are disposed. Specifically, as described above, in some examples, a controller can activate the electrical fluid operation components (208) to perform any number of fluid operations such as fluid analysis, fluid agitation, and fluid movement, among others. Accordingly, a controller passes electrical signals to the electrical fluid operation components (208) via the electric traces (210). The electric traces (210) route the signal from outside the housing (102), through the housing (102) wall, and to the electrical fluid operation components (208).

Additionally, through these electric traces (210), the individual fluid operation cell (100) is selected and activated. For example, each fluid operation cell (100) includes a dedicated address. Accordingly, each fluid operational cell (100) can be individually activated by the controller. Specifically, a controller sends a communication signal in serial to all the fluid operation cells (100) in an array. Each cell (100) decodes the signal to determine whether the address in that communication signal calls it or another cell (100). The cell (100) with the right address will be selected.

The fluid operation cell (100) also includes a semiconductor material based substrate (212) disposed inside the housing (102). The semiconductor material-based substrate (212) includes electrical contacts that interface with the electric traces (210) to receive the electrical signals from the controller, and thereby activate the electrical fluid operation component (208). The semiconductor material based substrate (212) may be a silicon substrate, a III-V substrate, or other type of semiconductor material based substrate. A III-V substrate is obtained by combining group Ill elements with group V elements from the periodic table.

In some examples, the semiconductor material-based substrate (212) includes active circuitry devices. Examples of active circuitry devices include registers, analog-to-digital converters (ADC), digital-to-analog converters (DCA), and transimpedance amplifiers (TIA), which rely on transistors, resistors, capacitors, and inductors on a silicon chip. In one specific example, the semiconductor material-based substrate (212) is a complementary metal-oxide semiconductor (CMOS) substrate that includes the active circuitry devices. Using a CMOS substrate allows transistor-based circuitry to be implemented on-chip. That is the implementation of high quality transistors are difficult, if possible, to implement on a printed circuit board as surface mount technology is implemented. Moreover, the implementation of transistors on a printed circuit board are large and costly. Accordingly, using a CMOS substrate allows for control and sensing circuitry to be included on the substrate of the fluid operation cell (100) as the transistors and other components can be “grown” on a wafer-like substrate.

Examples of such active circuitry devices include the electrical fluid operation components (208) disposed within the substrate. Using such a substrate with the fluid operational components (208) disposed thereon is beneficial in that it allows for increased functions and control over the fluidic operational components.

Disposed on, or in, the substrate (212) is an electrical fluid operation component (208). An electrical fluid operation component (208) works by using an electrical signal to operate on the bypassing fluid. As a specific example, the electrical fluid operation component (208) may include a pair of electrodes that are capacitively coupled to each other through the fluid. An electrical signal such as a voltage can then be passed through the electrodes to determine a type of fluid, whether there is particulate matter intermixed with the fluid, a chemical composition of the fluid, etc. The electrical fluid operation components (208) also can perform other functions such as heating the fluid, for example via a resistor, sensing the fluid, for example via an electrode sensor, actuating the fluid for example by using a piezo-resistive device to move fluid throughout the fluid operation cell (100), or to change a direction of fluid through the fluid operation cell (100). Other examples of fluid operations that can be carried via the application of an electrical signal include filtering, pumping, mixing, and spectroscopy.

The fluid operation cell (100) as described herein expands the capability of microfluidic systems. Specifically, each fluid operation cell (100) is an independent functional unit, and can perform a particular function without being coupled to another fluid operation cell (100). In other words, each fluid operation cell (100) is a complete package relying on just a voltage and control to activate the fluid operation cell (100). In other configurations, the cells in a system operate dependently to carry out a single operation. Moreover, as each cell (100) is a complete package with passages (104-1, 104-2) on the various surfaces, the modular cells can be arrayed in any orientation or configuration with other cells, even in three-dimensional arrangements to form complex and complete microfluidic operational systems.

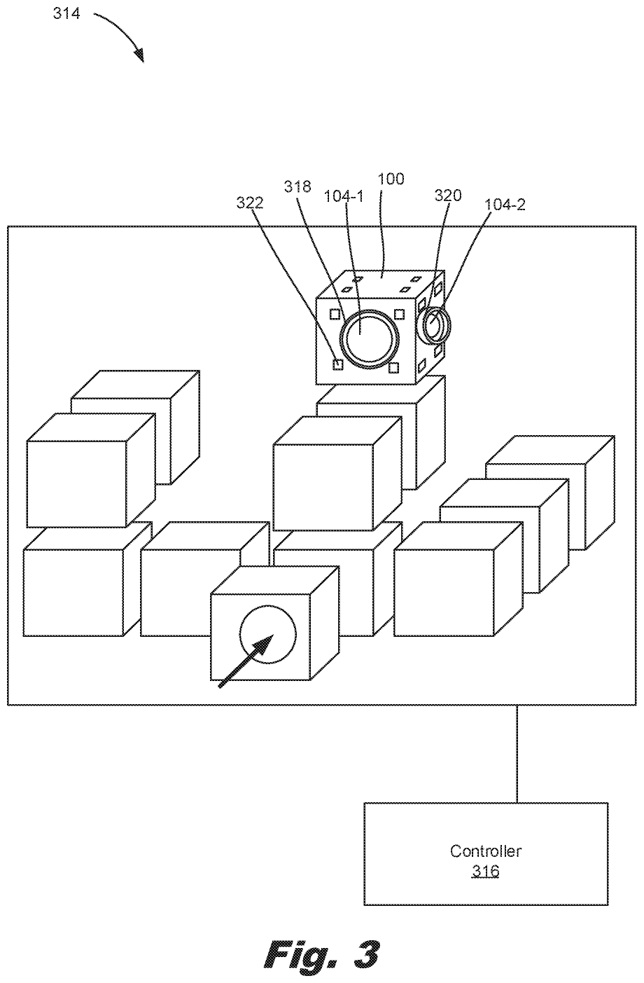

FIG. 3 is a diagram of a fluid operation system (314) with a fluid operation cell (100) with on-chip electrical fluid operation components (FIG. 2, 208), according to an example of the principles described herein. The fluid operation system (314) includes a number of fluid operation cells (100). For simplicity, just one of the fluid operation cells (100) includes a reference number. In the fluid operation system (314), each fluid operation cell (100) can carry out different functions. For example, one cell (100) may perform a heating operation, another may perform a temperature sensing operation, and yet another may perform a fluidic separating. Accordingly, an array of electric fluid operation cells (100) can be tailored to any desired form, i.e., to carry out any customized set of microfluidic operations.

In some examples, the group of fluid operation cells (100) may be divided into groups. A number of first fluid operation cells (100) may be the electrical fluid operation cells, meaning they rely on an electrical signal to carry out a fluidic operation. A number of second fluid operation cells of the system (314) may be non-electrical fluidic operational devices meaning they have fluidic operational components disposed within that do not rely on an electrical signal to carry out a fluidic operation.

As described above, the fluid operation cells (100) include a housing (FIG. 1, 102) having passages (104-1, 104-2) that allow the passage of fluid. As indicated in FIG. 3, the interconnection of the various fluid operation cells (100) may be such that fluid flows into and out of a particular fluid operation cell (100) on different surfaces as compared to others. This may be achieved by positioning passages (104) on multiple surfaces, and in some cases, all surfaces, of the fluid operation cells (100). With passages (104) disposed on different surfaces, any configuration or alignment of the number of fluid operation cells (100) is possible. Depending on the arrangement of fluid operation cells (100), some passages (104) may be unused. In these examples, a component of the fluid operation cell (100) may open/close a valve to unused passages (104) such that fluid does not escape. This is beneficial as it adds an extra dimension for arranging the cells (100) in the array. Specifically, in addition to a two-dimensional capability, i.e., left/right and front/back, an extra dimension, i.e., up/down, is available for designing the microfluidic system. Doing so increases the capabilities of fluid operation systems (314) and the ease of forming a fluid operation system (314).

As described above, each fluid operation cell (100) also includes an electrical fluid operation component (FIG. 2, 208) disposed within the housing (FIG. 1, 102). Each fluid operation cell (100) also include a connection system to interconnect with other of the number of fluid operation cells (100). This may include both mechanical and electrical connection systems. For example, each fluid operation cell (100) may include a male connector (320) to interconnect with a female connector (318) of another fluid operation cell (100). Each fluid operation cell (100) may also include a female connector (318) to interconnect with a male connector (320) of another fluid operation cell (100). In some example, these connectors (318, 320) may be integral to the passages (104) as protrusions or receiving holes.

Each fluid operation cell (100) may also include an electrical connection system. Specifically, the housing (FIG. 1, 102) of each fluid operation cell (100) may include electrodes (322) on the different surfaces to mate with, and route electrical signals from, the controller (316) to other of the number of fluid operation cells (100). For example, as described above, the controller (316) can pass any number of electrical signals to activate individual fluid operation cells (100). These signals are routed through the fluid operation system (314) via the electrical interconnection provided by these electrodes (322).

In addition to allowing mechanical and electrical connection between individual fluid operation cells (100), the mechanical and electrical connections allow the cells to be electrically and mechanically coupled to the controller (316).

The controller (316) of the fluid operation system (314) performs a number of functions. The controller may be a CPU, a GPU, an FPGA, or an ASIC which provides all digital and analog functions for digital signal processing, digital control, ADC, DAC, and TIA. Specifically, the controller (316) selects at least one fluid operation cell (100). This may be done by passing an electrical signal through the array of fluid operation cells (100) to the particular fluid operation cell (100) as described above. Through the same electrical connection, the controller (316) also activates the selected fluid operational cell (100). That is, the controller (316) may send an electrical signal with particular characteristics to activate the fluid operation cell (100). For example, if the fluid operation cell (100) is a heater, the controller (316) may pass a voltage of sufficient value to increase the heat of a resistor in the fluid operation cell (100).

The controller (316) in some examples also receives data from the selected at least one fluid operation cell (100). For example, if the fluid operation cell (100) is a capacitive sensor, the controller (316) may receive from the fluid operation cell data indicative of the current across the capacitive sensor. While specific examples have been provided regarding the activation and reception of data from particular types of fluid operation cells (100), any such activation may be performed and any type of data may be received.

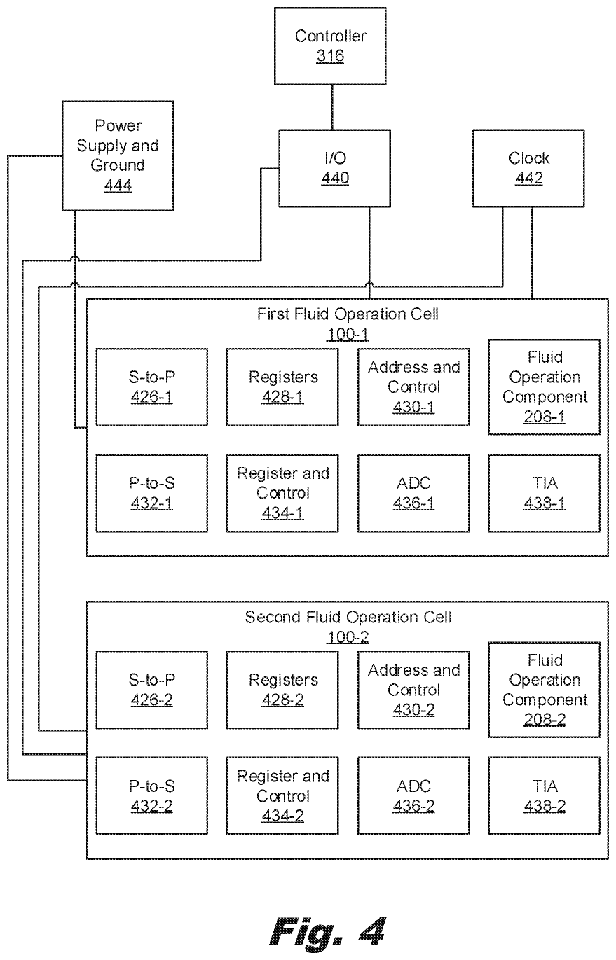

FIG. 4 is a schematic of the fluid operation system with a fluid operation cell (100) with on-chip electrical fluid operation components, according to an example of the principles described herein. In this example, the controller (316) receives instructions from a computing device on performing a particular operation. For example, the computing device may be the device that defines the different operations to be carried out on the subject fluid. The controller (316) then passes the control signal to the fluid operation cells (100-1, 100-2) via the I/O module (440).A power supply and ground module (444) provides electrical power to the fluid operation cells (100) and the clock module (442) synchronizes the passing of the different signals to the fluid operation cells (100).

As described above, the fluid operation cells (100) include various active circuit components to carry out an intended function. For example, a serial-to-parallel module (426-1, 426-2) converts incoming data signals from serial to parallel. A parallel-to-serial module (432-1, 432-2) performs an opposite action, i.e., converting an incoming signal from parallel to serial. The analog-to-digital converters (436-1, 436-2) convert analog signals to digital signals, and the trans-impedance amplifiers (438-1, 438-2) convert current to voltage. The registers (428-1, 428-2) are circuit components that store information. The address and control modules (430-1, 430-2) assist in the addressing and address searching for the fluid operation cells (100) and the register and control modules (434-1, 434-2) read and write the content of the registers (428-1, 428-2). Each fluid operation cell (100-1, 100-2) also includes a fluid operation component (208-1, 208-2) such as those described above.

The controller (316) is coupled to each of the fluid operation cells (100-1, 100-2, 100-3) either directly or indirectly through other fluid operation cells (100) as described above in connection with FIG. 3. It should be noted that while a single line is used to illustrate a connection between the fluid operation cells (100) and the controller, the single line does not represent a single physical connection, but is used to represent a logical connection. There may be multiple connections between the fluid operation cell (100) and the controller. Specifically, the connection between a fluid operation cell (00) and the controller (316) may include a power bus and a data bus.

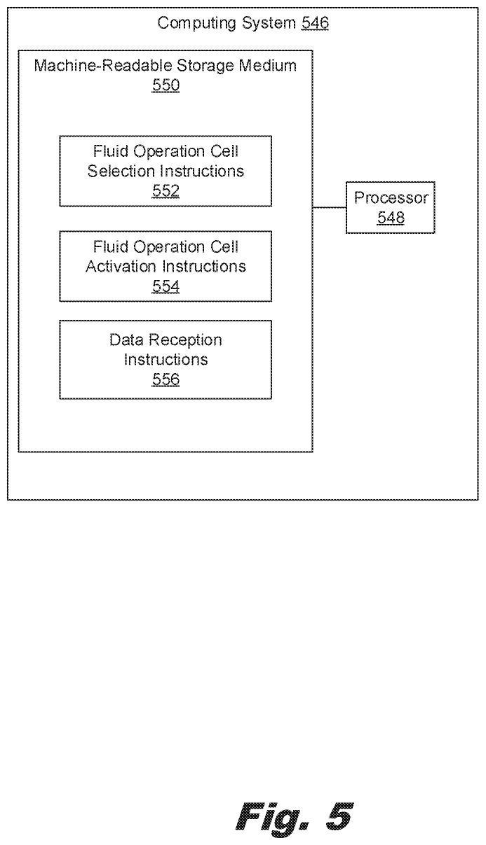

FIG. 5 is a block diagram of a computing system (546) for controlling a fluid operation cell (FIG. 1, 100) with on-chip electrical fluid operation components, according to an example of the principles described herein. To achieve its desired functionality, the computing system (546) includes various hardware components. Specifically, the computing system (546) includes a processor (548) and a machine-readable storage medium (550). The machine-readable storage medium (550) is communicatively coupled to the processor (548). The machine-readable storage medium (550) includes a number of instruction sets (552, 554, 556) for performing a designated function. The machine-readable storage medium (550) causes the processor (548) to execute the designated function of the instruction sets (552, 554, 556).

Although the following descriptions refer to a single processor (548) and a single machine-readable storage medium (550), the descriptions may also apply to a computing system (546) with multiple processors and multiple machine-readable storage mediums. In such examples, the instruction sets (552, 554, 556) may be distributed (e.g., stored) across multiple machine-readable storage mediums and the instructions may be distributed (e.g., executed by) across multiple processors.

The processor (548) may include at least one processor and other resources used to process programmed instructions. For example, the processor (548) may be a number of central processing units (CPUs), microprocessors, and/or other hardware devices suitable for retrieval and execution of instructions stored in machine-readable storage medium (550). In the computing system (546) depicted in FIG. 5, the processor (548) may fetch, decode, and execute instructions (552, 554, 556) for controlling a fluid operation cell. In one example, the processor (548) may include a number of electronic circuits comprising a number of electronic components for performing the functionality of a number of the instructions in the machine-readable storage medium (550). With respect to the executable instruction, representations (e.g., boxes) described and shown herein, it should be understood that part or all of the executable instructions and/or electronic circuits included within one box may, in alternate examples, be included in a different box shown in the figures or in a different box not shown.

The machine-readable storage medium (550) represent generally any memory capable of storing data such as programmed instructions or data structures used by the computing system (546). The machine-readable storage medium (550) includes a machine-readable storage medium that contains machine-readable program code to cause tasks to be executed by the processor (548). The machine-readable storage medium (550) may be tangible and/or non-transitory storage medium. The machine-readable storage medium (550) may be any appropriate storage medium that is not a transmission storage medium. For example, the machine-readable storage medium (550) may be any electronic, magnetic, optical, or other physical storage device that stores executable instructions. Thus, machine-readable storage medium (550) may be, for example, Random Access Memory (RAM), a storage drive, an optical disc, and the like. The machine-readable storage medium (550) may be disposed within the computing system (546), as shown in FIG. 5. In this situation, the executable instructions may be “installed” on the computing system (546). In one example, the machine-readable storage medium (550) may be a portable, external or remote storage medium, for example, that allows the computing system (546) to download the instructions from the portable/external/remote storage medium. In this situation, the executable instructions may be part of an “installation package”. As described herein, the machine-readable storage medium (550) may be encoded with executable instructions for controlling a fluid operation cell.

Referring to FIG. 5, fluid operation cell selection instructions (552), when executed by a processor (548), may cause the computing system (546) to select at least one of a number of modular fluid operation cells (FIG. 1, 100). Fluid operation cell activation instructions (554), when executed by a processor (548), may cause the computing system (546) to activate the selected at least one modular fluid operation cell (FIG. 1, 100). Data reception instructions (556), when executed by a processor (548), may cause the computing system (546) to receive data from the selected at least one modular fluid operation cells (FIG. 1, 100) which may include analyze the received data.

In some examples, the processor (548) and machine-readable storage medium (550) are located within the same physical component, such as a server, or a network component. The machine-readable storage medium (550) may be part of the physical component's main memory, caches, registers, non-volatile memory, or elsewhere in the physical component's memory hierarchy. In one example, the machine-readable storage medium (550) may be in communication with the processor (548) over a network. Thus, the computing system (546) may be implemented on a user device, on a server, on a collection of servers, or combinations thereof.

The computing system (546) of FIG. 5 may be part of a general-purpose computer. However, in some examples, the computing system (546) is part of an application specific integrated circuit.

In one example, using such a fluid operation cell 1) can be used across a wide variety of industries; 2) provides a robust microfluidic system due to the modular nature of the devices; 3) provides additional functionality through the inclusion of electrical fluid operation components; and 4) can be tailored to perform any, and any number, of microfluidic operations. However, it is contemplated that the devices disclosed herein may address other matters and deficiencies in a number of technical areas.

Aspects of the present system and method are described herein with reference to flowchart illustrations and/or block diagrams of methods, apparatus (systems) and computer program products according to examples of the principles described herein. Each block of the flowchart illustrations and block diagrams, and combinations of blocks in the flowchart illustrations and block diagrams, may be implemented by computer usable program code. The computer usable program code may be provided to a processor of a general purpose computer, special purpose computer, or other programmable data processing apparatus to produce a machine, such that the computer usable program code, when executed via, for example, the processor (548) of the computing system (546) or other programmable data processing apparatus, implement the functions or acts specified in the flowchart and/or block diagram block or blocks. In one example, the computer usable program code may be embodied within a computer readable storage medium; the computer readable storage medium being part of the computer program product. In one example, the computer readable storage medium is a non-transitory computer readable medium.

The preceding description has been presented to illustrate and describe examples of the principles described. This description is not intended to be exhaustive or to limit these principles to any precise form disclosed. Many modifications and variations are possible in light of the above teaching.

What is claimed is:

1. A fluid operation cell comprising:
a microelectromechanical housing comprising:
passages through which fluid flows; and
electric traces disposed on an interior of the housing to couple to a controller;
a semiconductive substrate disposed inside, and electrically coupled to the housing, wherein:
the substrate comprises an on-chip electrical fluid operation component formed thereon;
the electrical fluid operation component uses an electrical signal to operate on bypassing fluid; and
the fluid operation cell includes a dedicated address to be individually activated by the controller.

2. The cell of claim 1, wherein the cell is a microfluidic operational device.

3. The cell of claim 1, wherein the housing further comprises a common electrical interface on an external surface to electrically interconnect the fluid operation cell with other fluid operation cells and the controller.

4. The cell of claim 1, wherein the housing further comprises a common mechanical interface on an external surface to mechanically interconnect the fluid operation cell with other fluid operation cells and the controller.

5. The cell of claim 1, wherein the housing is a printed housing.

6. The cell of claim 1, wherein the housing is an enclosed shape.

7. The cell of claim 1, wherein the substrate includes active circuitry devices.

8. The cell of claim 1, wherein the electrical fluid operation component is selected from the group consisting of a heater, a sensor, an actuator, a filter, a pump, a mixer, and a spectroscopy component.

9. A fluid operational system comprising:
a number of fluid operation cells, wherein a fluid operation cell comprises:
a housing having at least one inlet and at least one outlet through which fluid flows;
an electrical fluid operation component disposed within the housing; and
a connection system to interconnect with other of the number of fluid operation cells;
wherein each fluid operation cell is uniquely addressable; and
a controller to:
select at least one fluid operation cell;
activate the selected at least one fluid operation cell; and
receive data from the selected at least one fluid operation cell.

10. The fluid operational system of claim 9, wherein the connection system of a fluid operation cell comprises:
a male connector to interconnect with a female connector of another fluid operation cell; and
a female connector to interconnect with a male connector of another fluid operation cell.

11. The system of claim 9, wherein the housing comprises electrodes on an external surface to mate with, and route electrical signals from the controller to, other of the number of fluid operation cells.

12. The system of claim 9, wherein at least one of the electrical fluid operation cells changes a fluid flow through the fluid operational system.

13. The system of claim 9, further comprising a number of second fluid operation cells, wherein the second fluid operation cells include non-electrical fluid operation components disposed within a housing.

14. A computing system comprising:
a processor;
a machine-readable storage medium communicatively coupled to the processor; and
an instruction set stored on the machine-readable storage medium to cooperate with the processor to:
select at least one of a number of modular fluid operation cells;
activate the selected at least one modular fluid operation cell; and
receive data from the selected at least one modular fluid operation cell.

15. The system of claim 14, wherein the instruction set further cooperates with the processor to analyze the received data from the selected at least one interchangeable fluid operation cell.
